(19) United States
(12) Patent Application Publication
(10) Pub. No.: US 2023/0031607 A1
(43) Pub. Date: Feb. 2, 2023

(54) COMPOSITION FOR RESPIRATORY DISEASES

(71) Applicant: Sol Gersh, Bal Harbour, FL (US)

(72) Inventor: Sol Gersh, Bal Harbour, FL (US)

(21) Appl. No.: 17/749,586

(22) Filed: May 20, 2022

Related U.S. Application Data
(60) Provisional application No. 63/227,812, filed on Jul. 30, 2021.

(51) Int. Cl. A61K 31/198; A61K 31/122; A61K 36/53; A61K 36/534; A61K 36/61

(52) U.S. Cl. CPC A61K 31/198; A61K 31/122; A61K 36/53; A61K 36/534; A61K 36/61

(57) ABSTRACT

A composition for the treatment and providing quick relief for respiratory diseases, such as Flu and Asthma. The composition includes Glutathione; Mentha oil; Pyrroloquinoline quinone; Wintergreen oil; and Eucalyptus oil. The composition is administered through the nasal route.

### CROSS-REFERENCE TO RELATED APPLICATIONS

This application claims priority from a U.S. Provisional Patent Appl. No. 63/227,812 filed on Jul. 30, 2021, which is incorporated herein by reference in its entirety.

### FIELD OF INVENTION

The present invention relates to a composition of respiratory diseases, and more particularly, the present invention relates to a composition for the treatment of respiratory diseases.

### BACKGROUND

Several types of respiratory diseases affect humans, such as infections and allergies. Besides making a person sick, respiratory diseases can make people uncomfortable, irritated, and often restless. Viral infections are common respiratory diseases that affect many and often result in epidemics. There is no full-proof cure for viral infections available but only supportive treatments are provided. Similarly, for allergies such as chronic obstructive pulmonary disease (COPD) and Asthma, chiefly, supportive treatments are available. Because of the lack of a proper cure, people suffering from respiratory diseases/disorders often resort to alternative therapies, such as natural remedies for getting relief. Natural and herbal remedies are often considered safer than synthetic drugs and are widely adopted by people for a number of diseases. However, the known natural medicines for respiratory disorders are very mild in action and often prove unsatisfactory.

A need is therefore appreciated for a novel composition for the treatment and relief of respiratory diseases that is potent and safe.

### SUMMARY OF THE INVENTION

The following presents a simplified summary of one or more embodiments of the present invention to provide a basic understanding of such embodiments. This summary is not an extensive overview of all contemplated embodiments and is intended to neither identify critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a composition for the treatment and relief of respiratory disorders.

It is another object of the present invention that the composition is safe to use.

It is still another object of the present invention that the composition can provide quick relief.

It is a further object of the present invention that children and adults can use the composition.

It is yet another object of the present invention that the composition is natural and organic.

In one aspect, disclosed is a composition that includes Glutathione, Mentha oil, Pyrroloquinoline quinone, Wintergreen oil, and Eucalyptus oil. The composition can be used for the treatment and relief of respiratory diseases, such as viral infection, COPD, Asthma, and the like.

### DETAILED DESCRIPTION

Subject matter will now be described more fully hereinafter. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, reasonably broad scope for claimed or covered subject matter is intended. Among other things, for example, the subject matter may be embodied as compositions or methods of treatment. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is to describe particular embodiments only and is not intended to be limiting to embodiments of the invention. As used herein, the singular forms “a”, “an”, and “the” are intended to include the plural forms as well, unless the context indicates otherwise. It will be further understood that the terms “comprise”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

Unless otherwise indicated, all numbers expressing quantities of ingredients used in this disclosure are to be understood as being modified in all instances by the term “about.” Accordingly, unless indicated to the contrary, the numerical parameters set forth in this disclosure are approximations that may vary depending upon the desired properties sought to be obtained by the present disclosure. At the very least, and not as an attempt to limit the application of the doctrine of equivalents to the scope of any claims, each numerical parameter should be construed in light of the number of significant digits and ordinary rounding approaches.

“Pharmaceutical composition” or “compositions” hereinafter refers to preparations that are in such a form as to permit the biological activity of the active agents to be unequivocally effective, and which contain no additional components which are toxic as administered to the patients.

“Subject” hereinafter refers to an individual and preferably a human who needs either relief and/or treatment. As used herein, a “subject” is the same as a “patient,” and the terms can be used interchangeably.

‘Treatment” hereinafter refers to medical treatment and includes both therapeutic and relief treatment depending on the context of use herein. “Treatment” includes reducing the virus load, preventing, or reducing complications, and symptomatic relief.

“Relief”: refers to a decrease in intensity of symptoms associated with medical conditions.

Disclosed is a composition for the treatment and relief of respiratory diseases, such as the diseases caused by infections and allergies. The disclosed composition can provide quick relief in viral infections, such as Flu and Covid infections, and hastens recovery by promoting natural defense mechanisms of the body. The disclosed composition can also provide relief in allergies-related respiratory diseases, such as COPD and Asthma. The disclosed composition can be administered through the nasal route in subjects in need thereof for the treatment and providing relief for respiratory diseases.

In certain implementations, the disclosed composition can include Glutathione, Mentha oil, Pyrroloquinoline quinone, Wintergreen oil, and Eucalyptus oil.

Glutathione is a tripeptide antioxidant that can protect underlying tissue from reactive oxygen species.

Mentha oil can be obtained from the herb Mentha arvensis using suitable methods. Preferably, the Mentha oil can be of high purity, such as about 100% pure. Any known methods for producing Mentha oil from the herb are within the scope of the present invention.

Pyrroloquinoline quinone (PPQ), also known as methoxatin, is a redox cofactor and antioxidant.

Wintergreen oil is an essential oil obtained from herbs using suitable processes, such as steam distillation. Any methods for extracting Wintergreen oil from herbs are within the scope of the present invention. Preferably, the Wintergreen oil of high purity can be used, such as about 100% pure Wintergreen oil.

Eucalyptus oil is an essential oil that can be obtained from the herb Eucalyptus Radiata. Eucalyptus oil obtained from Eucalyptus Globulus is also within the scope of the present invention. Eucalyptus oil of high purity can be used, such as about 100% pure Eucalyptus oil. Eucalyptus oil can be obtained from herbs using suitable processes, such as steam distillation, and such processes are within the scope of the present invention.

In certain implementations, the Glutathione can be present in the composition in a range of about 40-50%. Mentha oil can be present in the composition in a range of about 15-25%. Pyrroloquinoline quinone can be present in the composition in a range of about 10-20%. Wintergreen oil can be present in the composition in a range of about 5-15%. Eucalyptus oil can be present in the composition in a range of about 5-15%.

Preferably, the composition can include about 45% of Glutathione, about 20% of Mentha oil, about 15% of Pyrroloquinoline quinone, about 10% of Wintergreen oil, and about 10% of Eucalyptus oil.

In certain implementations, the disclosed composition can be prepared by mixing at least the ingredients including Glutathione, Mentha oil, Pyrroloquinoline quinone, Wintergreen oil, and Eucalyptus oil. The disclosed composition can also include neutral excipients for the formulation, and any such neutral excipients are within the scope of the present invention. The disclosed composition can be administered through the nasal route. A suitable formulation for nasal administration, such as for the atomizers can be formulated from the disclosed composition. Similarly, the formulations for inhalers can be formulated using the disclosed composition. The excipients and processes for nasal formulations are known to a skilled person, and any such formulation for nasal administration and the process of formulating the composition for nasal administration are within the scope of the present invention. Moreover, the dose for children and adults can be determined by suitable experimentation. Any such methods for determining the dose for children and adults are within the scope of the present invention.

What is claimed is:

1. A composition for treatment and relief of respiratory diseases, the composition comprising:
Glutathione;
Mentha oil;
Pyrroloquinoline quinone;
Wintergreen oil; and
Eucalyptus oil.

2. The composition according to claim 1, wherein:
Glutathione is present in an amount ranging from 15-25%;
Mentha oil is present in an amount ranging from 15-25%;
Pyrroloquinoline quinone is present in an amount ranging from 10-20%;
Wintergreen oil is present in an amount ranging from 5-15%; and
Eucalyptus oil is present in an amount ranging from 5-15%.

3. The composition according to claim 1, wherein:
Glutathione is present in an amount of 45%;
Mentha oil is present in an amount of 20%;
Pyrroloquinoline quinone is present in an amount of 15%;
Wintergreen oil is present in an amount of 10%; and
Eucalyptus oil is present in an amount of 10%.

4. A nasal dosage form for treatment and relief of respiratory diseases, the nasal dosage form comprises:
Glutathione;
Mentha oil;
Pyrroloquinoline quinone;
Wintergreen oil; and
Eucalyptus oil.

5. The nasal dosage form according to claim 4, wherein:
Glutathione is present in an amount ranging from 15-25%;
Mentha oil is present in an amount ranging from 15-25%;
Pyrroloquinoline quinone is present in an amount ranging from 10-20%;
Wintergreen oil is present in an amount ranging from 5-15%; and
Eucalyptus oil is present in an amount ranging from 5-15%.

6. The nasal dosage form according to claim 4, wherein:
Glutathione is present in an amount of 45%;
Mentha oil is present in an amount of 20%;
Pyrroloquinoline quinone is present in an amount of 15%;
Wintergreen oil is present in an amount of 10%; and
Eucalyptus oil is present in an amount of 10%.

7. A method for treatment and providing relief in respiratory diseases, the method comprises the steps of:
providing a composition comprising Glutathione, Mentha oil, Pyrroloquinoline, quinone, Wintergreen oil, and Eucalyptus oil; and
administering the composition through a nasal route.

8. The method according to claim 7, wherein:
Glutathione is present in an amount ranging from 15-25%;
Mentha oil is present in an amount ranging from 15-25%;
Pyrroloquinoline quinone is present in an amount ranging from 10-20%;
Wintergreen oil is present in an amount ranging from 5-15%; and
Eucalyptus oil is present in an amount ranging from 5-15%.

9. The method according to claim 7, wherein:
Glutathione is present in an amount of 45%;
Mentha oil is present in an amount of 20%;
Pyrroloquinoline quinone is present in an amount of 15%;
Wintergreen oil is present in an amount of 10%; and
Eucalyptus oil is present in an amount of 10%.
